(19) United States
(12) Patent Application Publication
(10) Pub. No.: US 2016/0263170 A1
(43) Pub. Date: Sep. 15, 2016

(54) NATURAL SWEETENER

(71) Applicant: NATGOOD IP, LLC, Houston, TX (US)

(72) Inventors: Ryan Turner, Houston, TX (US); Warren Nehmer, Houston, TX (US)

(73) Assignee: NATGOOD IP, LLC, Houston, TX (US)

(21) Appl. No.: 15/158,709

(22) Filed: May 19, 2016

Related U.S. Application Data
(63) Continuation-in-part of application No. 14/610,647, filed on Jan. 30, 2015.
(60) Provisional application No. 62/048,617, filed on Sep. 10, 2014.
(60) Provisional application No. 61/933,673, filed on Jan. 30, 2014.

(51) Int. Cl. A61K 36/28; A61K 8/97; A61K 8/73; A61K 31/723; A61K 8/34; A61K 31/047; A61K 31/7004; A61Q 11/00; A61K 8/60

(52) U.S. Cl. CPC A61K 36/28; A23L 1/097; A61Q 11/00; A61K 8/97; A61K 8/73; A61K 8/60; A61K 8/345; A61K 31/047; A61K 31/7004; A61K 31/723; A61K 2800/592

(57) ABSTRACT

A repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition. A sweetener composition prepared by: (i) dry blending in a fluidized bed a rheology modifier, tagatose, stevia extract, xanthan gum, a flavor blend, erythritol, and a second sugar alcohol to form a first mixture; (ii) spray depositing water onto the first mixture to generate a wet mixture; and (iii) removing excess moisture from the wet mixture to generate granules.

### TECHNICAL FIELD

This disclosure relates to sweetener compositions. More specifically this disclosure relates to compositions for use as natural sweeteners.

### BACKGROUND

Today artificial sweeteners and other sugar substitutes are found in a variety of food and beverages marketed as “sugar-free” or “diet,” including soft drinks, chewing gum, baked goods, candy, fruit juice, ice cream, and yogurt. Sugar substitutes are loosely considered any sweetener used in place of regular table sugar (sucrose).

Artificial sweeteners are regulated by the Food and Drug Administration (FDA) as food additives. They must be reviewed and approved by the FDA before being made available for sale. In some cases, the FDA declares a substance “generally recognized as safe” (GRAS). These GRAS substances, including highly refined stevia preparations, are deemed by qualified professionals based on scientific data as being safe for their intended use, or they have such a lengthy history of common use in food that they're considered generally safe and don't require FDA approval before sale.

Natural sweeteners are touted as offering a healthful alternative to artificial sweeteners but current products have a variety of challenges associated with their use including caloric content, taste, and appearance. Thus, an ongoing need exists for natural sweeteners having desirable characteristics.

### SUMMARY

Disclosed herein is a repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition.

Also disclosed herein is a sweetener composition prepared by (i) dry blending in a fluidized bed a rheology modifier, tagatose, stevia extract, xanthan gum, a flavor blend, erythritol, and a second sugar alcohol to form a first mixture; (ii) spray depositing water onto the first mixture to generate a wet mixture; and (iii) removing excess moisture from the wet mixture to generate granules.

### BRIEF DESCRIPTION OF THE DRAWINGS

FIG. 1 depicts plots of the time-intensity curves of common sweeteners and sucrose.

FIG. 2 depicts plots of the change in blood glucose level as a function of time for both a sweetener composition and sugar.

FIG. 3 is a plot of the change in blood glucose level as a function of time following ingestion of the sweetener compositions of example 2.

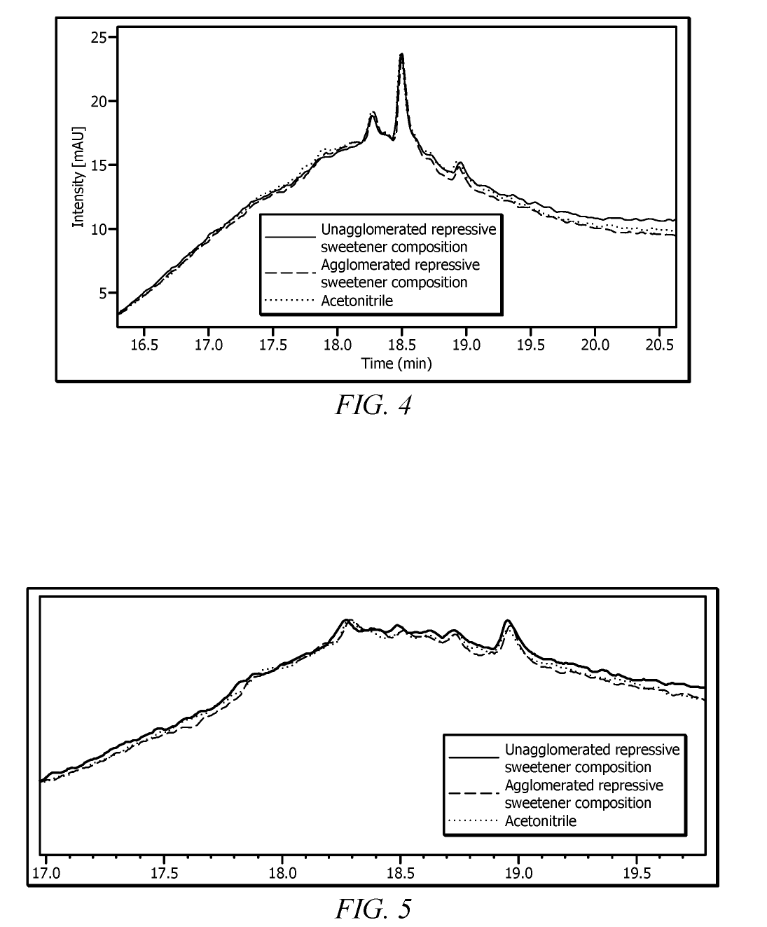

FIGS. 4 and 5 are overlays of the UV spectrum for the unagglomerated NSC, the agglomerated NSC, and acetonitrile (green).

FIGS. 6-8 are electrospray ionization mass spectra for the samples of Example 4.

FIGS. 9A and 9B are a comparison of the electrospray ionization mass spectra for the samples of Example 4.

FIG. 10 is an embodiment of an electrospray ionization mass spectra of a repressive sweetener composition of the type disclosed herein.

### DETAILED DESCRIPTION

Disclosed herein are natural sweetener compositions (NSCs). Also disclosed herein are ingestible compositions (both edible and nonedible) comprising the NSC. In an embodiment, the NSC comprises at least one stevia extract, at least one monk fruit extract, at least two sugar alcohols, and tagatose. An NSC of the type disclosed herein may optionally further comprise a blend of one or more natural flavors (i.e., a flavor blend). In some embodiments, the NSC further comprises a rheology modifier.

In an embodiment, the compositions disclosed herein are characterized by a low glycemic index when compared to table sugar (i.e., sucrose). The NSCs disclosed herein may also be characterized by the ability to attenuate the blood glucose response to consumption of other carbohydrate-containing foods and as such are characterized as repressive sweetener compositions.

In an embodiment, the NSC comprises at least one extract of stevia. Herein, the term extract refers to a preparation containing the active ingredient of a substance in a concentrated form. Stevia extract is known to be a source of steviol glycosides which are responsible for the sweet taste of the leaves of the stevia plant (Stevia rebaudiana). Steviol glycosides range in sweetness from 40 to 300 times sweeter than sucrose and are also characterized as being heat-stable, pH-stable, and non-fermentable. In an embodiment, the NSC comprises stevia in an amount of from about 0.01 weight percent (wt.)% to about 3 wt. %, alternatively from about 0.02 wt. % to about 1.8 wt. %, or alternatively from about 0.05 wt. % to about 0.8 wt. % based on the total weight of the NSC. The stevia may be a component of an extract where the stevia is present in densities ranging from 0.1 g/ml to 20 g/ml, alternatively about 1 g/ml to 5 g/ml or alternatively about 1 g/ml.

In an embodiment, the NSC comprises at least one monk fruit extract. Monk fruit is obtained from the plant Siraitia grosvenorii which is a herbaceous perennial vine of the Cucurbitaceae (gourd) family, native to southern China and northern Thailand. In an embodiment, the NSC comprises monk fruit n an amount of from about 0.05 wt. % to about 4 wt. %, alternatively from about 0.09 wt. % to about 3 wt. %, or alternatively from about 0.3 wt. % to about 2 wt. % based on the total weight of the NSC. The monk fruit may be a component of an extract where the monk fruit is present in densities ranging from 0.1 g/ml to 20 g/ml, alternatively about 1 g/ml to 5 g/ml or alternatively about 1 g/ml.

In an embodiment, the NSC comprises at least two sugar alcohols. Sugar alcohols, also known as polyols, polyhydric alcohols, or polyalcohols, are the hydrogenated forms of the aldoses or ketoses of a sugar. For example, glucitol, also known as sorbitol, has the same linear structure as the chain form of glucose, but the aldehyde (—CHO) group is replaced with a —CH₂OH group. Other common sugar alcohols include the monosaccharides erythritol and xylitol and the disaccharide maltitol. In an embodiment, the sugar alcohol comprises erythritol, mannitol, sorbitol, arabitol, threitol, xylitotl, ribitol, galactitol, fruitol, iditol, inositol, volemitol, lactitol, malitol, or combinations thereof. In an embodiment, the sugar alcohol comprises maltitol. In an embodiment, the sugar alcohols are present in the NSC in a combined amount of from about 20 wt. % to about 80 wt. %, alternatively from about 30 wt. % to about 70 wt. %, or alternatively from about 38 wt. % to about 60 wt. % based on the total weight of the NSC.

In an embodiment, the NSC comprises tagatose. Tagatose, or (3S,4S,5R)-1,3,4,5,6-pentahydroxy-hexan-2-one, is a naturally-occurring monosaccharide with the chemical formula C₆H₁₂O₆ (depicted in Structure I).

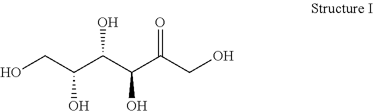 [SMILES: O=C(CO)[C@@H](O)[C@@H](O)[C@H](O)CO]

Tagatose has been a GRAS consumable since 2001. In an embodiment, tagatose is present in the NSC in an amount of from about 20 wt. % to about 95 wt. %, alternatively from about 30 wt. % to about 70 wt. %, or alternatively from about 40 wt. % to about 60 wt. % based on the total weight of the NSC.

The NSC may optionally comprise a blend of at least two flavors (i.e., a flavor blend) to create an enhancing natural flavor profile. Nonlimiting examples of natural flavors suitable for use in the flavor blend include oils or extracts having flavors such as thaumatin; citrus fruits; berries; orange; lemon; lime; tangerine; mandarin; grapefruit; acerola; grape; pear; passion fruit; pineapple; banana; apple; cranberry; cherry; raspberry; chokeberry; peach; plum; currant; black currant; cranberry; blackberry; pomegranate; acai; noni; elderberry; goji berry; rosehips; bilberry; hawthome berry; ginko; goru kola; rooibos; boysenberry; catuaba; horny goat weed; yohimbe; damiana; red raspberry leaf; vitex berry; blessed thistle; wolfberry; strawberry; mirabelle; watermelon; honeydew; cantaloupe; mango; papaya; botanical flavors derived from cola; tea; coffee; vanilla; almond; vegetables; tomato; cabbage; celery; cucumber; spinach; carrot; lettuce; watercress; dandelion; rhubarb; beet; cocona; cocoa; guava; Japanese knotweed; han guo, green tea, white tea, and any available tea blends; grapeseed; blueberries; or combinations thereof. In an embodiment, the flavor blend is included in the NSC any amount suitable to meet some user and/or process need. For example, the amount of flavor blend included may be designed to provide a desired organoleptic effect. In an embodiment, the flavor blend is present in the NSC an amount of from about 0.001 wt. % to about 0.095 wt. %, alternatively from about 0.002 wt. % to about 0.050 wt. %, or alternatively from about 0.005 wt. % to about 0.010 wt. % based on the total weight of the NSC.

In an embodiment, the NSC further comprises a rheology modifier. A rheology modifier suitable for inclusion in the NSC may comprise any material compatible with the other components of the NSC and able to provide the compositional properties disclosed herein. In an embodiment, the rheology modifier comprises a polysaccharide, alternatively a polysaccharide gum. A polysaccharide gum suitable for use in the present disclosure may be further characterized as being hydrocolloidal. Herein the term “hydrocollodial” takes on its standard meaning of a substance that forms a gel in the presence of water.

A polysaccharide gum suitable for use in the NSC may be derived from natural sources or may be prepared synthetically. In an embodiment, the polysaccharide gum comprises alginate, carrageenan, xanthan, pectin, gellan, welan, pullulan, curdlan, rhamsan, sphingan polymers, or combinations thereof. In an embodiment, the polysaccharide gum may be plant-derived. For example, carrageenan can extracted from certain species of the class Rhodophyceae (red seaweed) while xanthan gum, for example, is a polysaccharide that can be produced by fermentation using Xanthomonas bacterium. Gellan gum is the generic name of a polysaccharide that can be produced by cultured Pseudomonas elodea or related organisms. Curdlan is a polysaccharide gum that can be produced by a microorganism (e.g., Alcaligenes faecalis varmyxogenes). Alginate is a polysaccharide gum that can be obtained by extraction from certain species of seaweed, or alternatively Azotobacter vinelandii or Pseudomonas aeruginosa can be used to produce the polysaccharide gum through fermentation. In an embodiment, the polysaccharide gum comprises xanthan gum.

The backbone of the xanthan gum structure consists of two β-D-glucose units linked through the 1 and 4 positions. The side chain consists of two mannose and one glucuronic acid, so the chain consists of repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. In an embodiment, xanthan gum suitable for use in the present disclosure exhibits pseudoplastic behavior which is observable a wide range of temperatures (e.g., 90° C. to 260° C.). Herein pseudoplasticity is a term that refers to the reduction of viscosity when shear forces are applied.

In an embodiment, the rheology modifier is present in the NSC in an amount ranging from about 0.01 wt. % to about 3 wt. %, alternatively from about 0.05 wt. % to about 2 wt. %, or alternatively from about 0.1 wt. % to about 1 wt. % based on the total weight of the NSC.

In an embodiment, the components of an NSC of the type disclosed herein may be combined in any order and the composition prepared using any suitable methodology. Alternatively, an NSC of the type disclosed herein is formed into a particulate/powder material using techniques such as fluid bed agglomeration. Herein, fluid bed agglomeration refers to a process involving suspension of particles in an air stream followed by the spraying of a liquid onto the fluidized bed (top-down spray). Particles in the path of the spray develop an adhesive quality (i.e., become sticky) and upon collision with other particles in the bed adhere and form granules. The process may be continued until the granules develop some user and/or process desired characteristics.

In an embodiment, the NSC may be prepared by the mixing of all components of the NSC with the exception of the flavor blend in the fluidized bed. Agglomeration of the NSC may be accomplished by the spraying of the flavor blend onto the fluidized particles. For example, an NSC may be prepared by (i) dry blending in a fluidized bed a rheology modifier, tagatose, stevia extract, xanthan gum, a flavor blend, erythritol, and a second sugar alcohol to form a first mixture; (ii) spray depositing an aqueous solution (e.g., aqueous flavor blend) onto the first mixture to generate a wet mixture; and (iii) removing excess moisture from the wet mixture to generate granules.

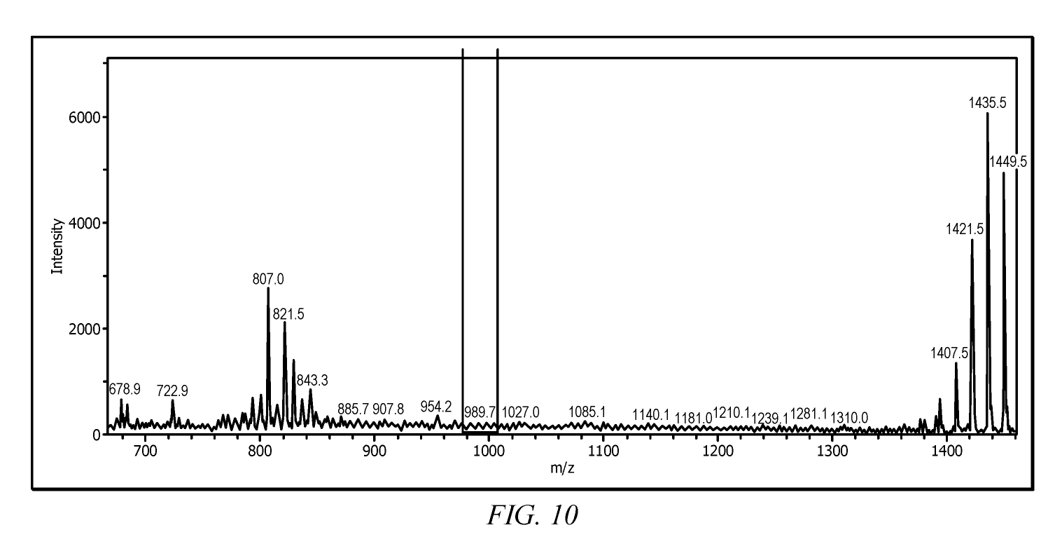

Further, an NSC of the type disclosed herein may form agglomerated particles having a characteristic fragmentation pattern when subjected to electrospray ionization (ESI) mass spectrometry. In an embodiment, an NSC when subjected ESI mass spectrometry is characterized by a mass spectrum with a mass to charge ratio (m/z) in the range of about 960 to 990 that has a signal intensity reduced by greater than about 90% when compared to the mass spectrum of an unagglomerated NSC in the same m/z range. Alternatively, an agglomerated NSC of the type disclosed herein is characterized by an electrospray ionization mass spectrum with a mass to charge ratio (m/z) in the range of about 960 to 990 that has a signal intensity reduced by greater than about 95% when compared to the mass spectrum of an unagglomerated NSC in the same m/z range. Alternatively, an NSC of the type disclosed herein has a signal intensity in the m/z range of about 960 to 990 that is ±10% of the baseline spectrum alternatively ±7%, or alternatively ±5%. In an embodiment, an NSC of the type disclosed herein has the electrospray mass ionization spectrum of FIG. 10.

In an embodiment, the NSC may be subjected to fluid bed agglomeration until a particular particle size distribution of the composition is reached. For example, fluid bed agglomeration of the NSC may be carried out to provide a mean particle size distribution ranging from about 250 microns to about 700 microns, alternatively from about 275 microns to about 500 microns, or alternatively from about 300 microns to about 400 microns. In an embodiment, the NSC may comprise a maximum of about 25% of particles having a mean particle size of less than about 200 microns. Further, the NSC may comprise a maximum of about 25% of particles having a mean particle size of greater than about 800 microns. NSCs of the type disclosed herein are characterized by a multimodal particle size distribution having a uniformity within each modality of the multimodal particle size distribution of ±20%, alternatively ±15%, or alternatively ±10%. Herein, a “multimodal” particle size distribution refers to the NSC having a plot of the cumulative volume (or indicator thereof) versus particle size characterized by the presence of more than one maxima or “mode” corresponding to different particle size ranges. In an embodiment, the NSC has a bimodal particle size distribution.

Particle size distribution of the NSC may impact manufacturing of the materials from the production and processing side to the appearance of the product to the user. An NSC having the disclosed particle size features may be characterized by the rapid dissolution of particles in hot and cold systems, reduced separation of the NSC particle when it is a component of a dry mix, reduced dust generation, and the presence of larger particle sizes that provides a positive visual impact.

In an embodiment, the NSC has an apparent density, also known as a bulk density, comparable to that of table sugar. Herein, the apparent density refers to the mass of a unit volume of the NSC, which can be freely (without pressing or tapping) poured into a container. An NSC of the type disclosed herein may be characterized by an apparent density that is within about ±30% of the apparent density of table sugar, alternatively about ±20% of the apparent density of table sugar, or alternatively about ±10% of the apparent density of table sugar. For example, the apparent density of the NSC may be from about 750 kg/m³ to about 940 kg/m³, alternatively from about 775 kg/m³ to about 925 kg/m³, or alternatively from about 800 kg/m³ to about 875 kg/m³ and be determined in accordance with ISO 697.

In an embodiment, the NSC has a compressibility that is increased when compared to that of table sugar. Herein, the compressibility refers to the degree to which the NSC powder compacts as a function of applied pressure. The compressibility of an NSC can be evaluated by calculating the compressibility index. The compressibility index is determined by measuring the unsettled apparent volume, Vₒ, and the final tapped volume of the material (i.e., NSC), V_{f}, after tapping the material until no further volume change occurs. The compressibility index is then calculated using the equation:

Compressibility index=100%×(Vₒ−V_{f}/Vₒ)

The NSC may be characterized by a compressibility that is about 10% greater than the compressibility of table sugar, alternatively that is about 20% greater than the compressibility of table sugar, or alternatively that is about 30% greater than the compressibility of table sugar. In an embodiment, an NSC has a compressibility index of from about 1% to about 20%, alternatively from about 5% to about 20%, or alternatively from about 5% to about 10%.

In an embodiment, the NSC has a flowability comparable to that of table sugar. Herein, the flowability refers to the ability of the NSC to flow through a calibrated orifice. For example, flowability is a consideration when using high-speed rotary tablet machines, in order to ensure homogenous and rapid flow of material for uniform die filling. During the short dwell-time (milliseconds) used in these high speed rotary tablet machines, the required amount of material should be transferred into the die cavities with a reproducibility of ±5%. The Hausner ratio is a metric for evaluating the flowability of a material and is calculated by dividing Vₒ by V_{f}. Hausner ratios less than 1.25 indicate a free flowing material. An NSC may be characterized by a flowability that is within about ±30% of the flowability of table sugar, alternatively about ±20% of the flowability of table sugar, or alternatively about ±10% of the flowability of table sugar. Further, the NSC may have a Hausner ratio of from about 0.1 to about 1.2, alternatively from about 0.5 to about 1.0, or alternatively from about 0.5 to about 0.75.

In an embodiment, the NSC has a sugar recipe equivalence (SRE) of about 1. Herein, the SRE refers to the amount of NSC equivalent to the amount of table sugar in a particular recipe. For example, a SRE of about 1 indicates that for a recipe using 1 cup of table sugar, 1 cup of an NSC may be substituted. In an embodiment, the NSC has an SRE of about 0.85, alternatively about 0.90, or alternatively 0.95.

In an embodiment, the NSC has a moisture retention value comparable to that of table sugar. Herein, the moisture retention value refers to the extent to which the NSC binds water in an ingestible product. An NSC of the type disclosed herein may be characterized by a moisture retention value that is within about ±30% of the moisture retention value of table sugar, alternatively about ±20% of the moisture retention value of table sugar, or alternatively about ±10% of the moisture retention value of table sugar.

In an embodiment, the NSC has a rise ratio of about 1. Herein, the rise ratio refers to the extent to which a baked good rises when prepared with table sugar in comparison to the rise of the same baked good in the presence of the NSC. In an embodiment, the NSC has a rise ratio of about 0.85, alternatively about 0.90, or alternatively 0.95.

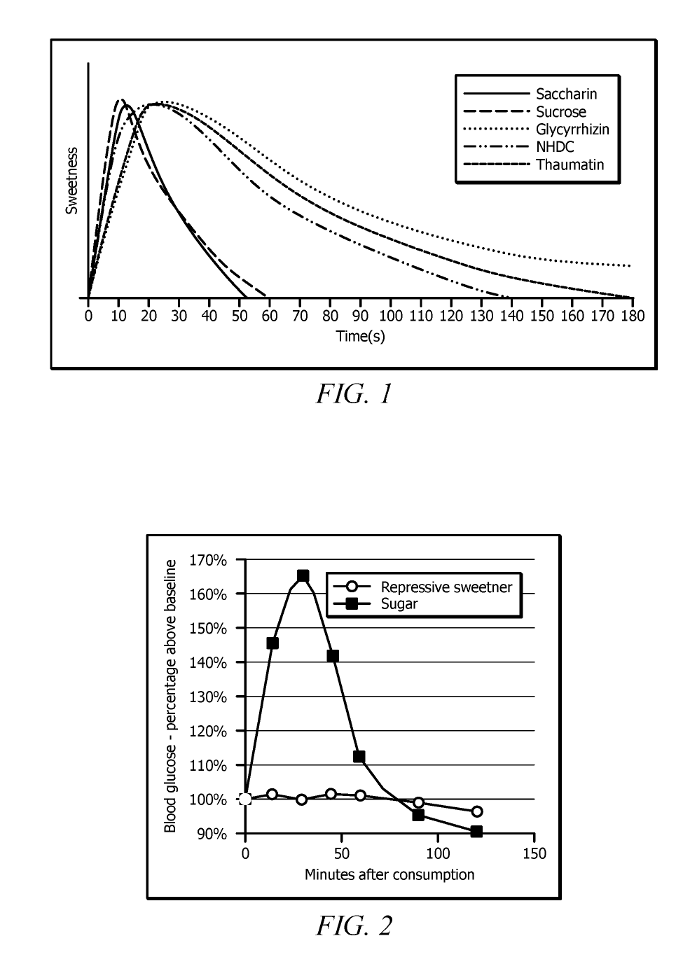

In an embodiment, the NSC has a time-intensity curve that is similar to that of sucrose. Herein, the intensity refers to the intensity of sweetness. The time-intensity curves for common sweeteners and sucrose are presented in FIG. 1. The time intensity curve for the NSC may be sufficiently similar to that of sucrose so as allow formulations with citric acid that balance sweet and sour taste profiles. Sweetness intensity can be determined by descriptive testing which is a sensory analysis using human assessors (e.g., an expert panel) to evaluate a particular feature of an ingestible product. For example the flavor attributes of the sample (e.g., sweetness) are tracked via an appropriate computer program for a set time period following ingestion of a sample and the information used to prepare a time intensity profile.

In an embodiment, an NSC of the type disclosed herein may be utilized as a sugar source in a fermentation process.

In an embodiment, the NSC is characterized by a low glycemic index. Herein, the glycemic index (GI) refers to a number associated with carbohydrate-containing foods that indicates the food's effect on a person's blood glucose (also called blood sugar) level. A value of 100 represents the standard, an equivalent amount of pure glucose. The GI estimates how much each gram of available carbohydrate (total carbohydrate minus fiber) in a food raises a person's blood glucose level following consumption of the food, relative to consumption of pure glucose.

Glycemic index can be measured by any suitable methodology. For example, to determine a food's GI value, measured portions of the food containing 50 grams of available carbohydrate (or 25 grams of available carbohydrate for foods that contain lower amounts of carbohydrate) may be fed to 10 healthy people after an overnight fast. Finger-prick blood samples can be taken at 15-30 minute intervals over the next two hours. These blood samples can be used to construct a blood sugar response curve for the two hour period. The incremental area under the curve (iAUC) is calculated to reflect the total rise in blood glucose levels after eating the test food. The GI value is calculated by dividing the iAUC for the test food by the iAUC for the reference food (same amount of glucose) and multiplying by 100. The average of the GI ratings from all ten subjects is designated as the GI for that food. In an embodiment, an NSC of the type disclosed herein has a GI value of from about 1 to about 10, alternatively from about 2 to about 7, or alternatively from about 2 to about 5 based on consumption of 50 g of available carbohydrates.

In an embodiment, an NSC of the type disclosed herein is able to attenuate the blood glucose response of a subject that ingests carbohydrate-containing foods (CCF) subsequent to ingestion of the NSC. For example, from about 1 minute to about 3 hours subsequent to ingestion of an NSC of the type disclosed herein, ingestion of another CCF may produce a blood glucose response that is reduced by from about 5% to about 90% when compared to the blood glucose response produced without prior ingestion of the NSC. The repression of the blood glucose response subsequent to the ingestion of the NSC is predicated upon the ingestion of an effective amount of the NSC and may continue for a period of time ranging from about 5 minutes to about 2 hours or alternatively from about 15 minutes to about 1 hour following ingestion of the NSC. In some embodiments, when compared to the blood glucose response of the CCF ingested without prior ingestion of an NSC of the type disclosed herein, the blood glucose response after ingestion of the NSC is reduced by from about 10% to about 80%, or alternatively from about 20% to about 50%. The NSCs disclosed herein act as repressive sweetener compositions which reduce the blood glucose level response to ingestion of a CCF to the ranges disclosed herein.

In an embodiment, an NSC of the type disclosed herein is characterized by a GI for the composition as a whole that is less than the sum of the GI values of the constituents (taking into account their weight percentage in the composition). An example of a NSC of the type disclosed herein is a composition comprising maltitol, erythritol, tagatose, monkfruit, a rheology modifier, stevia, and a flavor blend, and is characterized by a GI of from about 2 to about 6.

In an embodiment, an NSC of the type disclosed herein is included in an edible composition such as a food product. “Food” herein refers to any edible product intended for consumption by humans or animals, including solids, semi-solids, or liquids (e.g., beverages). The term “food” and the term “food and beverage” are herein used interchangeably. Examples of food and beverage products or formulations include, but are not limited to sweet coatings, frostings, or glazes for comestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionary category, the Dairy Product category, the Ice Cream category, the Meal Replacement category, the Pasta and Noodle category, the Sauces, Dressings, Condiments category, the Baby Food category, and/or the Spreads category.

In general, the Soup category refers to canned/preserved, dehydrated, instant, chilled, ultra-high temperature (UHT) and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

“Dehydrated and Culinary Food Category” herein refers to: (i) Cooking aid products such as: powders, granules, pastes, concentrated liquid products, including concentrated bouillon, bouillon and bouillon-like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid, or frozen.

The Beverage category herein refers to beverages, beverage mixes and concentrates, including but not limited to, carbonated and non-carbonated beverages, alcoholic and non-alcoholic beverages, ready-to-drink beverages, liquid concentrate formulations for preparing beverages such as sodas, and dry powdered beverage precursor mixes. The Beverage category also includes alcoholic drinks, soft drinks, sports drinks, isotonic beverages, and hot drinks. The alcoholic drinks include, but are not limited to beer, cider/perry, flavored alcoholic beverages, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The hot drinks include, but are not limited to coffee, such as fresh, instant, and combined coffee; tea, such as black, green, white, oolong, and flavored tea; and other hot drinks including flavor-, malt- or plant-based powders, granules, blocks or tablets mixed with milk or water.

The Snack Food category generally refers to any food that can be a light informal meal including, but not limited to sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream, sugar and flavoring. Examples of ice cream include, but are not limited to: impulse ice cream; take-home ice cream; frozen yogurt and artisanal ice cream; soy, oat, bean (e.g., red bean and mung bean), and rice-based ice creams.

The Confectionary category generally refers to edible products that are sweet to the taste. Examples of confectionary include, but are not limited to candies, gelatins, chocolate confectionery, sugar confectionery, gum, and the likes and any combination products.

The Meal Replacement category generally refers to any food intended to replace the normal meals, particularly for people having health and/or fitness concerns. Examples of meal replacements include, but are not limited to slimming products and convalescence products.

The Ready Meal category generally refers to any food that can be served as a meal without extensive preparation or processing. The ready meal category includes products that have had recipe “skills” added to them by the manufacturer, resulting in a high degree of readiness, completion and convenience. Examples of ready meals include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles.

The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to honey, chocolate spreads, nut-based spreads, and yeast-based spreads.

The Dairy Product category generally refers to edible products produced from mammal's milk. Examples of dairy product include, but are not limited to drinking milk products, cheese, yogurt and sour milk drinks, and other dairy products.

Exemplary foods include confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, powder mints, boiled sweets, pastilles, chews, toffees, caramels and nougat, medicated confectionery, lollipops, liquorice, other sugar confectionery, gum, chewing gum, sugarized gum, sugar-free gum, functional gum, bubble gum, cough drops, herbs, seeds, spices, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, ready-to-eat cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, fruit juices, vegetable juices, cream, cheese, processed cheese, spreadable processed cheese, unspreadable processed cheese, unprocessed cheese, spreadable unprocessed cheese, hard cheese, packaged hard cheese, unpackaged hard cheese, yogurt, plain/natural yogurt, flavored yogurt, fruited yogurt, probiotic yogurt, drinking yogurt, regular drinking yogurt, probiotic drinking yogurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded cereals and snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, yeast-based spreads, toppings, and syrups.

In one embodiment, foods include alcoholic beverages, baby food, baby formula, baked goods, breakfast cereals, cheese, chewing gum, coffee whiteners, condiments and relishes, confectionary and frostings, crackers, dairy products, egg products, fats and oils, fish products, frozen dairy, frozen dinners, fruit ices, gelatins and puddings, grain mixtures, granulated sugar, imitation dairy products, coffee, coffee products and coffee beverages, jams and jellies, meat products, milk products, non-alcoholic beverages, nut products, grains and grain products, poultry, processed fruits, processed vegetables, reconstituted vegetables, ready to eat meals, salad dressings, seasonings and flavors, snack foods, soft candy, soups, sugar substitutes, sweet sauce, sweetener blends, table top sweeteners, tea, tea products, and tea beverages.

In an embodiment, foods include table top sweeteners and beverages. Beverages include, but are not limited to, fruit juices, soft drinks, tea, coffee, beverage mixes, milk drinks, alcoholic and nonalcoholic beverages.

In an embodiment, an NSC is included in a non-edible ingestible product. Herein, “non-edible ingestible products” refer to supplements, nutraceuticals, functional food products (e.g., any fresh or processed food claimed to have a health-promoting and/or disease-preventing properties beyond the basic nutritional function of supplying nutrients), pharmaceutical and over the counter products, oral care products such as dentifrices and mouthwashes, cosmetic products such as sweetened lip balms and other personal care products that use sucralose and/or other sweeteners. In general, over the counter (OTC) product and oral care product generally refer to product for household and/or personal use which may be sold without a prescription and/or without a visit to a medical professional. Examples of the OTC products include, but are not limited to vitamins and dietary supplements; topical analgesics and/or anesthetic; cough, cold and allergy remedies; antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g. muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral care products include, but are not limited to mouth cleaning strips, toothpaste, toothbrushes, mouthwashes/dental rinses, denture care, mouth fresheners, at-home teeth whiteners and dental floss.

In an embodiment, a food product comprises the NSC. In such embodiments, the food product may be utilized after introduction of the NSC to the food product with no additional heat-treatment of the food product. In another embodiment, the food product may be heat-treated (e.g., baked) subsequent to the addition of the NSC.

In an embodiment, an NSC of the type disclosed herein is characterized by a great taste, a reduced cooling effect, a low glycemic index, and exertion of positive effects on the digestive health of the subject ingesting the NSC or an ingestible product comprising the NSC. In an embodiment, the NSC exhibits a reduced cooling effect when dissolved in a solvent such as water. Herein, the cooling effect refers to the endothermic solvation of the composition.

In an embodiment, the NSC is a component of an ingestible product (e.g., edible) and exerts a positive effect on the digestive health of the subject consuming the ingestible product. In an embodiment, the NSC is a prebiotic. Herein, prebiotic refers to non-digestible food ingredients that stimulate the growth and/or activity of beneficial bacterial flora of the gastrointestinal tract. Many digestive disorders are caused by unfavorable alterations in the beneficial bacterial flora of the gastrointestinal tract. The result is a significant disruption of intestinal lining integrity due to a proliferation of pathogens such as E. coli, Clostridium perfringens, H. pylori, etc. This imbalance in microflora, or dysbiosis, is most commonly caused by the use of antibiotics that can wipe out both good and bad bacteria. Other causative factors are GI infections (e.g., traveler's diarrhea), certain drugs such as acid-suppressing medications, chronic mal-digestion, chronic constipation, stress, and diet. A primary role of beneficial gastrointestinal microflora is to help protect the gut lining Once these microflora become imbalanced, the host's immune capabilities become compromised thus leading to more serious digestive disorders such as inflammatory bowel disease (IBD) which include Crohn's disease (CD) and ulcerative colitis (UC). In an embodiment, an NSC of the type disclosed herein is a component of a treatment designed to prevent or ameliorate conditions associated with a gastrointestinal disorder such as Crohn's disease or UC.

In an embodiment, the NSC may also affect blood glucose levels and have a beneficial effect on insulin sensitivity. These characteristics may be useful in the management of one or more disorders or dysfunction. For example, the NSC and compositions comprising the NSC may facilitate the control of diabetes and/or lower LDL blood cholesterol by interfering with the absorption of dietary cholesterol.

In an embodiment, an NSC of the type disclosed herein may be a component of a weight-loss treatment or regimen. The prevalence of obesity among adults in the USA in 2008 was 68% (˜200 million people). Even a modest degree of obesity, particularly if the excess fat is located in the abdomen, increases the risks for type 2 diabetes mellitus cardiovascular disease, stroke, and some forms of cancer, In an embodiment, NSC of the type disclosed herein and compositions comprising the NSC may be utilized as part of a regimen designed to achieve or maintain a user-determined weight and/or body mass index.

In another embodiment, an NSC of the type disclosed herein may be utilized in ingestible products to reduce rapid decreases in blood sugar levels, also termed a “sugar crash.” For example, a typical diet in industrialized nations contains a large amount of simple carbohydrates such as maltodextrin or sugars such as sucrose, fructose, glucose, and dextrose. This type of diet often results in gastrointestinal stress and dehydration due to the high osmotic pressure of the smaller molecules in the stomach leading to dramatic and detrimental swings in blood sugar levels as sugar rapidly enters the blood. The result is the induction of an insulin response that drives down sugar levels, resulting in a sugar crash where blood sugar levels dip below the level prior to consumption of the fuel, thereby exacerbating the effects of non-diabetic hypoglycemia or low blood sugar. In an embodiment, an NSC of the type disclosed herein is a component of a regimen designed to reduce the incidence of non-diabetic hypoglycemia in a subject.

In another embodiment, the NSC may be a component of a treatment for the prevention or reduction of dental caries. Caries is a multi-factorial disease that involves prolonged colonization of acid-producing bacteria on teeth. Fermentation of dietary carbohydrates by caries leads to a localized drop in pH below a critical value of 5.5, resulting in the demineralization of enamel and potentially damaging underlying tooth structure. If left untreated, caries can lead to the progressive destruction of tooth structure, pain, tooth loss, loss of oral function, as well as have systemic health consequences. In an embodiment, an NSC of the type disclosed herein is a component of a regimen designed to reduce the incidence of dental caries in a subject.

In each aspect of the utilization of an NSC of the type disclosed herein as a component in the treatment of a disease or dysfunction, it is contemplated the subject will be administered and/or supplied an ingestible product comprising the NSC. The NSC may partially or fully replace sugar in said product and thus the subject is given a reduced calorie, ingestible material as a component of the treatment. It is contemplated that the treatment may also comprise conventional methodologies. For example, for obesity the treatment in addition to comprising ingestion of the NSC may further comprise exercise and other activities or compositions that function to promote weight loss. Thus, in an embodiment, a methodology of the type disclosed herein further comprises the ingestion of the NSC in conjunction with one or more conventional therapies for addressing the particular disorder or dysfunction.

In an aspect, the present disclosure comprises a blend of an NSC and an artificial sweetener. The blend may contain a ratio of NSC:artificial sweetener ranging from 1:10 to 10:1, alternatively from 3:7 to 7:3, or alternatively 1:1. Examples of artificial sweeteners suitable for use in the blend include without limitation acesulfame potassium, aspartame, neotame, sucralose, advantame, and combinations thereof.

While embodiments of the present disclosure have been shown and described, modifications thereof can be made by one skilled in the art without departing from the spirit and teachings of the disclosure. The embodiments described herein are exemplary only, and are not intended to be limiting. Many variations and modifications of the disclosure are possible and are within the scope of the invention. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Background is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed it is intended to disclose or claim individually each possible number that such a range could reasonably encompass, including any sub-ranges encompassed therein. When describing a range of measurements every possible number that such a range could reasonably encompass can, for example, refer to values within the range with one significant digit more than is present in the end points of a range. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to proviso out or exclude any individual members of any such group, including any sub-ranges or combinations of sub-ranges within the group, if for any reason Applicants choose to claim less than the full measure of the disclosure.

### ADDITIONAL DISCLOSURE

The disclosed subject matter is a repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition.

An additional aspect of the disclosed subject matter alone or in addition to any one or all of the described aspects is the repressive sweetener composition formulated as agglomerated particles having a multimodal particle size distribution.

An additional aspect of the disclosed subject matter alone or in addition to any one or all of the described aspects is the repressive sweetener composition formulated to have a mean particle size distribution ranging from about 200 microns to about 700 microns.

An additional aspect of the disclosed subject matter alone or in addition to any one or all of the described aspects is the repressive sweetener composition comprising less than about 25% of particles having a mean particle size distribution of less than about 200 microns.

An additional aspect of the disclosed subject matter alone or in addition to any one or all of the described aspects is the repressive sweetener composition comprising less than about 25% of particles having a mean particle size of greater than about 800 microns.

An additional aspect of the disclosed subject matter alone or in addition to any one or all of the described aspects is the repressive sweetener composition having an apparent density ranging from about 750 kg/m³ to about 940 kg/m³.

An additional aspect of the disclosed subject matter alone or in addition to any one or all of the described aspects is the repressive sweetener composition having a compressibility index of from about 1% to about 20%.

An additional aspect of the disclosed subject matter alone or in addition to any one or all of the described aspects is the repressive sweetener composition having a Hausner ratio of from about 0.1 to about 1.2.

An additional aspect of the disclosed subject matter alone or in addition to any one or all of the described aspects is the repressive sweetener composition wherein the sugar alcohols comprise arabitol, erythritol, lactitol, maltitol, mannitol, sorbitol, xylitol, or combinations thereof.

An additional aspect of the disclosed subject matter alone or in addition to any one or all of the described aspects is the repressive sweetener composition wherein the rheology modifying additive comprise xanthan gum, gellan, gum Arabic, gum ghatti, agar, tragacanth gum, or combinations thereof.

An additional aspect of the disclosed subject matter alone or in addition to any one or all of the described aspects is the repressive sweetener composition wherein the flavor blend comprises at least two flavors selected from the group consisting of: thaumatin; berries; orange; lemon; lime; tangerine; mandarin; grapefruit; acerola; grape; pear; passion fruit; pineapple; banana; apple; cranberry; cherry; raspberry; chokeberry; grapeseed; peach; plum; currant; black currant; blackberry; blueberry; pomegranate; acai; noni; elderberry; goji berry; rosehips; bilberry; hawthome berry; ginko; goru kola; rooibos; boysenberry; catuaba; horny goat weed; yohimbe; damiana; red raspberry leaf; vitex berry; blessed thistle; wolfberry; strawberry; mirabelle; watermelon; honeydew; cantaloupe; mango; papaya; botanical flavors derived from cola; white tea; green tea; coffee; vanilla; almond; tomato; cabbage; celery; cucumber; spinach; carrot; lettuce; watercress; dandelion; rhubarb; beet; cocona; cocoa; guava; Japanese knotweed; lo han guo, and tea blends.

The disclosed subject matter is also a sweetener composition prepared by (i) dry blending in a fluidized bed a rheology modifier, tagatose, stevia extract, xanthan gum, a flavor blend, erythritol, and a second sugar alcohol to form a first mixture; (ii) spray depositing water onto the first mixture to generate a wet mixture; and (iii) removing excess moisture from the wet mixture to generate particles.

An additional aspect of the disclosed subject matter alone or in addition to any one or all of the described aspects is the sweetener composition wherein the electrospray ionization mass spectrum of the first mixture has a normalized signal intensity in the mass to charge ratio range of 960 to 992 that is 50% to 90% greater than a normalized signal intensity in the mass to charge ratio range of 960 to 992 for the crystals.

An additional aspect of the disclosed subject matter alone or in addition to any one or all of the described aspects is the sweetener composition having a multimodal particle size distribution.

An additional aspect of the disclosed subject matter alone or in addition to any one or all of the described aspects is the sweetener composition having a bimodal particle size distribution.

An additional aspect of the disclosed subject matter alone or in addition to any one or all of the described aspects is the sweetener composition having a glycemic index of from about 1 to about 10.

An additional aspect of the disclosed subject matter alone or in addition to any one or all of the described aspects is the sweetener composition wherein ingestion of a carbohydrate-containing food subsequent to ingestion of the composition produces a blood glucose response that is reduced by from about 5% to about 90% when compared to a blood glucose response produced without prior ingestion of the composition.

An additional aspect of the disclosed subject matter alone or in addition to any one or all of the described aspects is the sweetener composition wherein the second sugar alcohol comprises arabitol, lactitol, maltitol, mannitol, sorbitol, xylitol, or combinations thereof.

An additional aspect of the disclosed subject matter alone or in addition to any one or all of the described aspects is the sweetener composition further comprising sucrose.

An additional aspect of the disclosed subject matter alone or in addition to any one or all of the described aspects is the sweetener composition is an ingestible product comprising the sweetener composition.

An additional aspect of the disclosed subject matter alone or in addition to any one or all of the described aspects is a method of treating a subject comprising administering the ingestible product.

An additional aspect of the disclosed subject matter alone or in addition to any one or all of the described aspects is a method of treating a subject comprising administering the ingestible product wherein the subject has been diagnosed with a disorder selected from the group consisting of obesity, hypertension, dyslipidemia, type 2 diabetes, coronary heart disease, gallbladder disease, osteoarthritis, dental caries, gastrointestinal disorders, malignant neoplasms, and musculoskeletal disorders.

An additional aspect of the disclosed subject matter alone or in addition to any one or all of the described aspects is a prebiotic comprising a repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition.

An additional aspect of the disclosed subject matter alone or in addition to any one or all of the described aspects is a nutritional supplement comprising a repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition.

An additional aspect of the disclosed subject matter alone or in addition to any one or all of the described aspects is a comestible product comprising a repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition wherein the composition is present in an amount equivalent to an amount of table sugar utilized in an otherwise similar composition and wherein the repressive sweetener composition comprises maltitol.

An additional aspect of the disclosed subject matter alone or in addition to any one or all of the described aspects is formulation comprising the repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition and an artificial sweetener wherein the artificial sweetener comprises acesulfame potassium, aspartame, neotame, sucralose, advantame, or combinations thereof.

### EXAMPLES

The present disclosure is further illustrated by the following example, which is not to be construed in any way as imposing limitations upon the scope thereof. On the contrary, it is to be clearly understood that resort can be had to various other aspects, embodiments, modifications, and equivalents thereof which, after reading the description herein, can suggest themselves to one of ordinary skill in the art without departing from the spirit of the present invention or the scope of the appended claims.

The data and descriptions provided in the following example are given to show particular aspects and embodiments of the subject matter disclosed, and to demonstrate a number of the practices and advantages thereof. The example is given as a more detailed demonstration of some of the aspects and embodiment described herein and is not intended to limit the disclosure or claims in any manner.

### Example 1

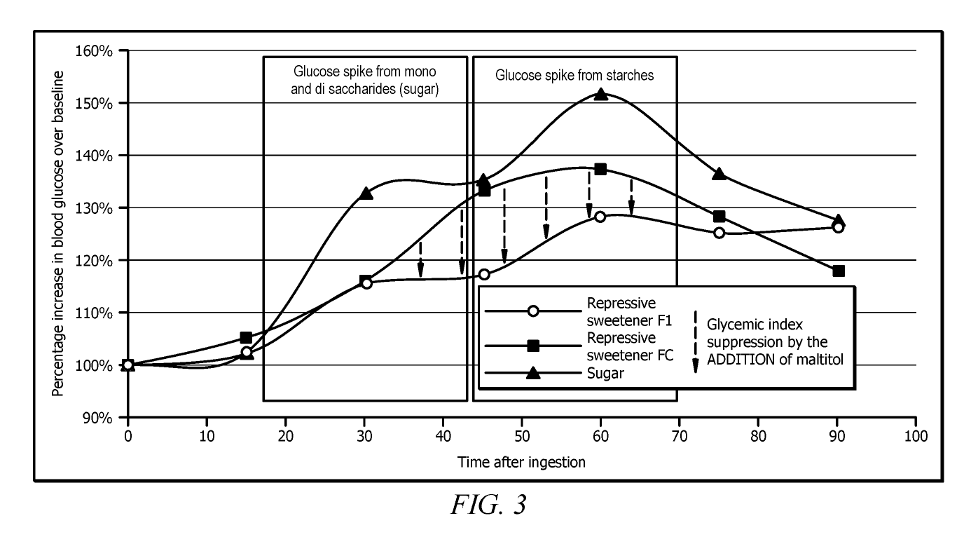

The blood glucose response to NSCs of the type disclosed herein was compared to that of table sugar. Specifically, an NSC containing erythritol, tagatose, maltitol, monkfruit, xanthum gum and natural flavors was formulated. Subjects' baseline blood glucose level was determined and set as 100% at time 0. The subjects then ingested a sufficient amount of NSC to provide 50 grams of carbohydrates and the blood glucose levels were determined at periodic intervals for up to 120 minutes after ingestion. A plot of the blood glucose level increase as a percentage above baseline for an NSC of the type disclosed herein is presented in FIG. 2. The blood glucose level change following the ingestion of sugar in an amount to provide 50 grams of carbohydrates is also shown in FIG. 2.

The results demonstrate an NSC of the type disclosed herein results in minimal changes in the blood glucose levels over the measured time period in comparison to table sugar.

### Example 2

The effect on the GI response for different NSC formulations was assessed. Four formulations, designated F1-F4, containing tagatose, monkfruit, stevia extract, erythritol, xanthan gum and either maltitol or lactitol were prepared as indicated in Table 1. The glycemic index for each formulation was calculated based on the glycemic index for the individual components taking into account the weight percent of the component in the formulation. Monkfruit, xanthan gum, and stevia extract have a glycemic index of 0, erythritol has a glycemic index of 1 while tagatose has a glycemic index of 3. Maltitol has a glycemic index of 35 while lactitol has a glycemic index of 5. Fifteen subjects were evaluated and the GI calculated as described previously herein.

TABLE 1

| Sample | Maltitol | Lactitol | Expected GI | Observed GI |
|---|---|---|---|---|
| F1 | YES | NO | 7.21 | 4.41 |
| F2 | NO | YES | 2.27 | 6.43 |
| F3 | YES | YES | 4.74 | 3.03 |
| F4 | NO | NO | 3.00 | 2.09 |
| SUGAR | NO | NO | 65 | 65 |

The results demonstrate that unexpectedly, maltitol despite having a much higher glycemic index than lactitol, 35 and 5 respectively; produced NSCs having a lower than expected overall glycemic index.

### Example 3

The effect of an NSC on the blood glucose level attenuation in the presence of a CCF was investigated. Specifically, a cake mix was prepared containing the ingredients listed in Table 2.

TABLE 2

| | Mass | | | % in dry mass | g per |
|---|---|---|---|---|---|
| Ingredient | (Quantity) | | Glycemic Index | (est.) | cupcake |
| 112 g flour | 112 | g | 71 extra carbs | 33.4% | 14 |
| 100 g sweetener or | 100 | g | 65 for sugar | 29.8% | 12.5 |
| sugar | | | <5 for NSC | | |
| ½ tsp baking | 2.84 | g (½ tsp) | 0 | 0.85% | 0.355 |
| powder | | | | | |
| ½ tsp baking soda | 2.84 | g (½ tsp) | 0 | 0.85% | 0.355 |
| ¼ tsp salt | 1.42 | g (¼ tsp) | 0 | 0.42% | 0.1775 |
| 60 ml buttermilk | 62.16 | g (60 ml) | 31 extra carbs | 18.53% | 7.77 |
| 70 g unsalted butter | 70 | g | 0 | 20.86% | 8.75 |
| 1 extra large egg | 56 | g | 0 | 16.69% | 7 |
| 1½ tsp vanilla | 6.5 | g | 0 | 1.94% | 0.81 |
| extract | | | | | |

| | |
|---|---|
| Total 413.7 g wet, 335.51 g dry (dry has a 19% moisture loss) | Total extra carbs = 34.27 g |
| Yield 8 cupcakes | |

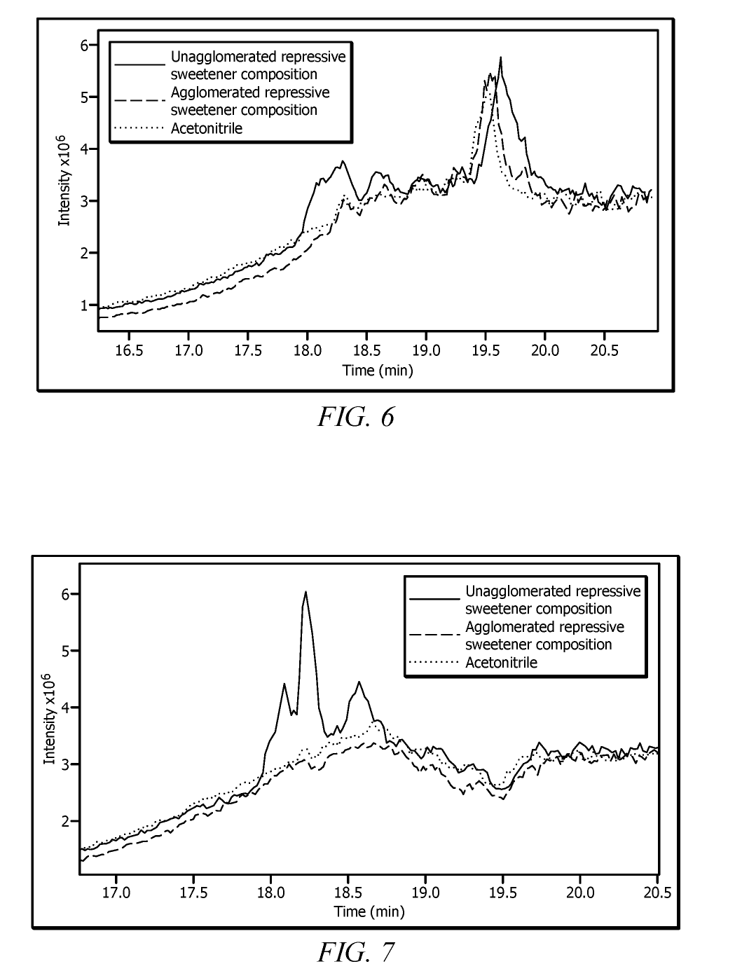

The dry mix contained 34.27 g of extra carbs. Cupcakes were prepared using either sugar, the F1 NSC from example 2 which had maltitol, or the F3 NSC from example 2 which contained both maltitol and lacitol. A plot of the percentage increase for _ subjects ingesting the cupcakes as a function of time is presented in FIG. 4. The data demonstrate that cupcakes prepared with the NSC F1 (i.e., containing maltitol) resulted in a lower blood glucose level increase over the baseline when compared to ingestion of cupcakes prepared with either sugar or the NSC F3 which had a combination of maltitol and lactitol. The data also demonstrate the suppressive effect of NSC F1 (i.e., containing maltitol) persists in the presence of other carbohydrate-containing ingredients.

### Example 4

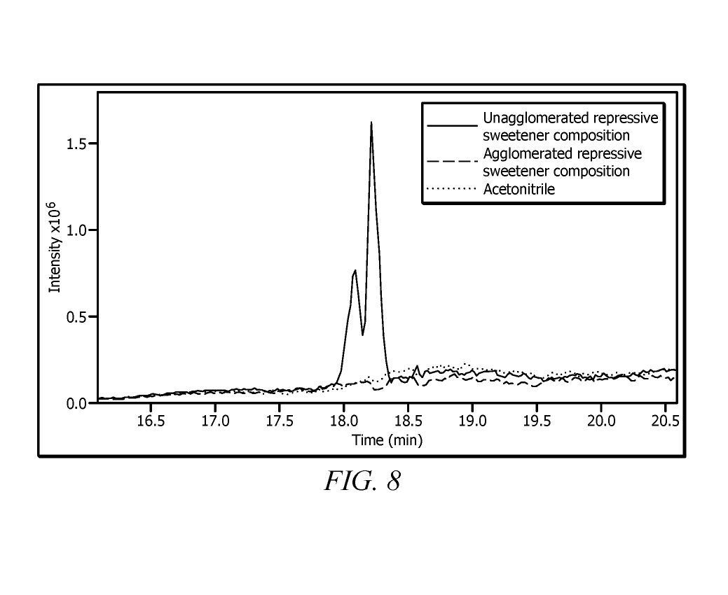

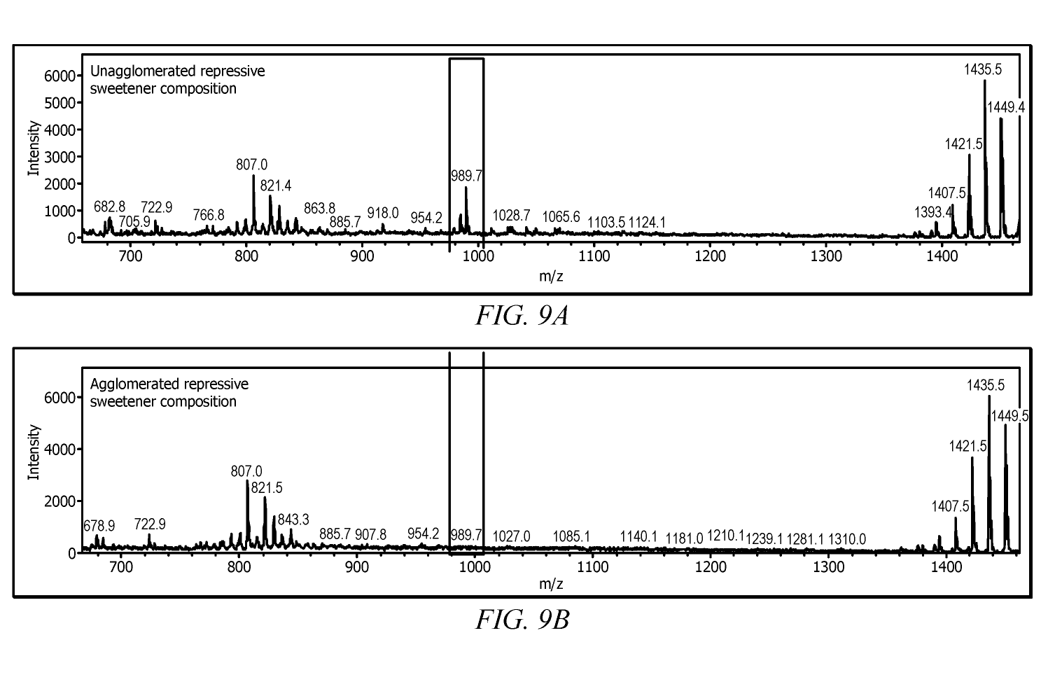

The ESI mass spectrum for an NSC was obtained. Specifically, each sample was weighed out (˜10 mg) and dissolved to a final concentration 10 mg/ml. The samples were then extracted using acetonitrile (ACN). Fisher Optima LCMS grade was used. The samples which were not fully soluble at that concentration were vortexed 15 seconds, waited, and vortexed again. Aliquots (100 μl) were removed for LCMS analysis and 8 μl injected onto an Agilent ZORBAX SB-C18 (2.1×100 mm) column. Solvent A was 5% ACN/94% H₂O/1% acetic acid while solvent B was Methanol 100%. The column was run at a flow rate of 200 μl/min using two gradients: (1) a slow gradient 5-60% B in 10 min, then to 100% B in 5 min, 3 min hold and (2) a steeper gradient 5-100% B in 15 min, 3 min hold (18 min total). Sample analysis was carried out in UV using the full spectrum 190-400 nm, and 215, 275 and 290 nm single wavelength chromatographic trace while the mass spectrum utilized a gentle source with a sensitivity optimized for m/z around 800 and the analysis was conducted in both negative and positive ionization mode. FIGS. 4 and 5 are overlays of the UV spectrum for the unagglomerated NSC (red), the agglomerated NSC (blue) and acetonitrile (green). Analysis of FIGS. 4 and 5 suggests there is little difference between the agglomerated and unagglomerated samples. Mass spectrometry of the samples in positive mode demonstrates a difference between the unagglomerated and agglomerated samples, FIGS. 6 and 7. Similar trends were observed when using the negative ESI mode, FIG. 8. A comparison of the LCMS traces between the unagglomerated and agglomerated NCS is shown in FIG. 9 which demonstrates a clear difference in the m/z range of 967 to 989.

### Example 5

A sweetener composition of the type disclosed herein (i.e., an NSC) will be compared to table sugar and other commercially available sweetners using 6 differentiating tests carried out on at least 10 members of the lay public, lacking specialized knowledge in sweeteners:

### 1) The Sugar Similarity Test

An amount of refined white sugar consisting of typical comparable granule size and fine content is placed into a bowl and compared one by one with other sugar substitutes, including the Natural Sweetener described herein. 10 members of the lay public, lacking specialized knowledge in sweeteners, are asked to compare the sugar versus the sugar substitute in the bowl and identify which is sugar and which is the sugar substitute based on appearance, smell and taste within a 1 minute period. A sweetener is considered to pass the test and be enough like sugar when 80% of the people testing fail to differentiate the sugar versus the sugar substitute based on appearance, smell and dipping their fingertip into the sugar or sugar substitute to taste.

### 2) The Yellow Cake-Bake Test

A cake that when baked has a light yellow hue is made from the following ingredients: 120 g flour, 150 g sugar or sweetener, ½ teaspoon baking powder, ½ teaspoon baking soda, ¼ teaspoon salt, 65 ml buttermilk, 30 g vegetable oil, 30 g unsalted butter (softened), 1 extra large egg, 1½ teaspoon vanilla extract. The oven is pre-heat to 300 F, flour is sifted, sugar or sweetener is added with baking powder, baking soda and salt into a large standing mixer bowl. In a separate bowl the egg, vanilla extract, buttermilk and vegetable oil are whisked together. Using the whisk attachment on the standing mixer the butter is added to the dry ingredients and mixed well on a low speed until well mixed. The liquid mixture is slowly added to the dry ingredients and once combined turn the mixer up to full speed and whisk for one minute until all the ingredients are homogeneous. The mixture is divided between 8 cupcake cases spaced out evenly in a muffin pan and place into the oven for 10 minutes. The pan is then turned and baked for a further 10 minutes. A skewer is inserted into the cakes to ensure they are done; the skewer coming out clean if the cakes are done.

A sweetener is considered to pass the test and be enough like sugar when a) 80% of the people testing fail to differentiate the cake made with sugar versus the cake made with sugar substitute based on appearance, smell, taste and mouth-feel within the same day of being baked b) 80% of the people testing fail to differentiate the cake made with sugar versus the cake made with sugar substitute based on appearance, smell, taste and mouth-feel 3 days after being stored in an open container (i.e. a moisture retention test).

### 3) The Caramelization Test

1 cup of caramelized sugar or 1 cup of sweetener is mixed with ½ cup of water and swirled to mix until it browns. A sweetener is considered to pass the test and be enough like sugar when it browns within 25% of the time when the sugar browns.

### 4) The Chocolate Bar Test

Chocolate bars are prepared by blending 200 g melted cocoa liquor, 250 g melted cocoa butter into running bench-top chocolate melangeur (“Santha” brand chocolate melangeur), once well blended 1 teaspoon of soya lecithin, 250 g dry powdered whole milk powder, 0.06% ground vanilla bean and 350 g sugar or sweetener and added gradually and then allowed to mélange for 1.5 days. The mix is then tempered, molded and cooled. A sweetener is considered to pass the test and be enough like sugar when 80% of the people testing fail to differentiate the chocolate bar made with sugar versus the sugar substitute made with sugar based on appearance, smell and mouth-feel. This test is designed to screen out sweeteners that produce a strong endothermic or cooling sensation when the chocolate is melted on the tongue.

### 5) The Tea Test

Iced tea is prepared using established methods and sugar or sweetener is used at twice the recommended concentration. A sweetener is considered to pass the test and be enough like sugar when 80% of the people testing fail to differentiate the Iced tea made with sugar versus Iced tea made with sugar substitute based on appearance, smell, taste and lack of unusual after-taste.

### 6) A Glycemic Response Test

Under an Institutional Review Board approved, controlled clinical trial, subjects undergo a pre-test fast for at least 20 hours and then consume 30 grams of glucose, or an equal amount of sweetener in water. Blood glucose measurements are then made every 15 minutes for 90 minutes and the blood glucose response is recorded. A sweetener is considered to pass the test when then blood glucose response is less than 5% of glucose.

### 7) A Repression Test

Under controlled study scenario subjects fast, then consume a product (e.g. a cupcake) that contains other nutritive carbohydrates and the glycemic response is assessed by established methods. The glycemic response to the nutritive carbohydrates is compared to the glycemic response to the nutritive carbohydrates in the presence of the sweetener or sugar. A sweetener is considered to pass the repression test when then blood glucose response is much lower than expected for the combination of nutritive carbohydrates and sweetener.

The results of these tests are summarized in Table 3.

TABLE 3

| | | Natural | A mix of sucralose, | | Erythritol, | |
|---|---|---|---|---|---|---|
| | | Sweetener | dextrose and | Rebaudioside A, | oligosaccharides, | Saccharin, |
| | | (described | maltodextrin | erythritol, | natural flavors | dextrose, |
| | Sugar | herein) | (Splenda) | natural flavors | (Swerve) | cream of tartar |
| Sugar similarity | PASS | PASS | FAIL | FAIL | FAIL | FAIL |
| test | | | | | | |
| Yellow cake-bake | PASS | PASS | FAIL | FAIL | FAIL | FAIL |
| test | | | | | | |
| Caramelization | PASS | PASS | | | | |
| test | | | | | | |
| Chocolate | PASS | PASS | FAIL | FAIL-AFTER | FAIL- | FAIL-AFTER |
| bar test | | | | TASTE | STRONG | TASTE |
| | | | | | COOLING | |
| | | | | | SENSATION | |
| Tea test | PASS | PASS | PASS | FAIL- | FAIL- | FAIL-AFTER |
| | | | | VANILLA | COOLING | TASTE |
| | | | | AFTER | AFTER | |
| | | | | TASTE | TASTE | |
| Glycemic test | FAIL | PASS | CONTAINS | PASS | PASS | CONTAINS |
| | | | DEXTROSE | | | DEXTROSE |
| Repression test | FAIL | PASS | FAIL | FAIL | FAIL | FAIL |

| | Dextrose, | | | Liquid drops | | |
|---|---|---|---|---|---|---|
| | Acesulfame-K, | | | made from a | | |
| | Cream of Tartar, | Dextrose, | Dextrose, | stevia leaf | Inulin, | Dextrose, |
| | Calcium Silicate, | Rebaudioside A, | maltodextrin, | extraction | stevia, | monkfruit |
| | Natural Flavors. | Natural Flavors | aspartame | process | silica | extract |
| Sugar similarity | FAIL | FAIL | FAIL | FAIL | FAIL | FAIL |
| test | | | | | | |
| Yellow cake-bake | FAIL | FAIL | FAIL | FAIL | FAIL | FAIL |
| test | | | | | | |
| Caramelization | | | | | | |
| test | | | | | | |
| Chocolate | FAIL-AFTER | FAIL-AFTER | FAIL-AFTER | FAIL-AFTER | FAIL-AFTER | FAIL-TASTE |
| bar test | TASTE | TASTE | TASTE | TASTE | TASTE | |
| Tea test | FAIL-AFTER | FAIL-AFTER | FAIL-AFTER | FAIL-AFTER | FAIL-NOT | FAIL-NOT |
| | TASTE | TASTE | TASTE | TASTE | SWEET | SWEET |
| | | | | | ENOUGH | ENOUGH |
| Glycemic test | CONTAINS | CONTAINS | CONTAINS | PASS | PASS | CONTAINS |
| | DEXTROSE | DEXTROSE | DEXTROSE | | | DEXTROSE |
| Repression test | FAIL | FAIL | FAIL | FAIL | FAIL | FAIL |

| | Tagatose, erythritol plus | | | | |
|---|---|---|---|---|---|
| | stevia, monkfruit or | | Isomalto- | | |
| | stevia and monkfruit | Erythritol | oligosaccahrides | Tagatose | Allulose |
| Sugar similarity | PASS | FAIL | FAIL | PARTIAL | PARTIAL |
| test | | | | (LESS | (LESS |
| | | | | SWEET) | SWEET) |
| Yellow cake-bake | FAIL-DOES | FAIL | FAIL | FAIL-TOO | PASS |
| test | NOT RETAIN | | | DARK | |
| | MOISTURE | | | BROWNS | |
| | | | | TOO FAST | |
| Caramelization | | | | FAIL-TOO | |
| test | | | | DARK | |
| | | | | BROWNS | |
| | | | | TOO FAST | |
| Chocolate | PASS | FAIL- | FAIL- | PARTIAL | PARTIAL |
| bar test | | STRONG | RHEOLOGY | (LESS | (LESS |
| | | COOLING | | SWEET) | SWEET) |
| | | SENSATION | | | |
| Tea test | PASS | FAIL-AFTER | FAIL-NOT | PARTIAL | PARTIAL |
| | | TASTE | SWEET | (LESS | (LESS |
| | | | ENOUGH | SWEET) | SWEET) |
| Glycemic test | PASS | PASS | FAIL | PASS | PASS |
| Repression test | FAIL | FAIL | FAIL | FAIL | PASS |

What is claimed is:

1. A repressive sweetener composition comprising at least two sugar alcohols, monk fruit, a rheology modifier, and tagatose wherein the repressive sweetener composition has a glycemic index that is less than a sum of the glycemic index of each component of the repressive sweetener composition.

2. The composition of claim 1 formulated as agglomerated particles having a multimodal particle size distribution.

3. The composition of claim 1 formulated to have a mean particle size distribution ranging from about 200 microns to about 700 microns.

4. The composition of claim 3 comprising less than about 25% of particles having a mean particle size distribution of less than about 200 microns.

5. The composition of claim 3 comprising less than about 25% of particles having a mean particle size of greater than about 800 microns.

6. The composition of claim 1 having an apparent density ranging from about 750 kg/m³ to about 940 kg/m³.

7. The composition of claim 1 having a compressibility index of from about 1% to about 20%.

8. The composition of claim 1 having a Hausner ratio of from about 0.1 to about 1.2.

9. The composition of claim 1 wherein the sugar alcohols comprise arabitol, erythritol, lactitol, maltitol, mannitol, sorbitol, xylitol, or combinations thereof.

10. The composition of claim 1 wherein the rheology modifying additive comprise xanthan gum, gellan, gum Arabic, gum ghatti, agar, tragacanth gum, or combinations thereof.

11. The composition of claim 1 wherein the flavor blend comprises at least two flavors selected from the group consisting of: thaumatin; berries; orange; lemon; lime; tangerine; mandarin; grapefruit; acerola; grape; pear; passion fruit; pineapple; banana; apple; cranberry; cherry; raspberry; chokeberry; grapeseed; peach; plum; currant; black currant; blackberry; blueberry; pomegranate; acai; noni; elderberry; goji berry; rosehips; bilberry; hawthome berry; ginko; goru kola; rooibos; boysenberry; catuaba; horny goat weed; yohimbe; damiana; red raspberry leaf; vitex berry; blessed thistle; wolfberry; strawberry; mirabelle; watermelon; honeydew; cantaloupe; mango; papaya; botanical flavors derived from cola; white tea; green tea; coffee; vanilla; almond; tomato; cabbage; celery; cucumber; spinach; carrot; lettuce; watercress; dandelion; rhubarb; beet; cocona; cocoa; guava; Japanese knotweed; lo han guo, and tea blends.

12. A sweetener composition prepared by:
(i) dry blending in a fluidized bed a rheology modifier, tagatose, stevia extract, xanthan gum, a flavor blend, erythritol, and a second sugar alcohol to form a first mixture;
(ii) spray depositing water onto the first mixture to generate a wet mixture; and
(iii) removing excess moisture from the wet mixture to generate particles.

13. The composition of claim 12 wherein the electrospray ionization mass spectrum of the first mixture has a normalized signal intensity in the mass to charge ratio range of 960 to 992 that is 50% to 90% greater than a normalized signal intensity in the mass to charge ratio range of 960 to 992 for the crystals.

14. The composition of claim 12 having a multimodal particle size distribution.

15. The composition of claim 12 having a bimodal particle size distribution.

16. The composition of claim 12 having a glycemic index of from about 1 to about 10.

17. The composition of claim 12 wherein ingestion of a carbohydrate-containing food subsequent to ingestion of the composition produces a blood glucose response that is reduced by from about 5% to about 90% when compared to a blood glucose response produced without prior ingestion of the composition.

18. The composition of claim 12 wherein the second sugar alcohol comprises arabitol, lactitol, maltitol, mannitol, sorbitol, xylitol, or combinations thereof.

19. The composition of claim 12 further comprising sucrose.

20. An ingestible product comprising the sweetener composition of claim 12.

21. A method of treating a subject comprising administering the ingestible product of claim 20.

22. The method of claim 21 wherein the subject has been diagnosed with a disorder selected from the group consisting of obesity, hypertension, dyslipidemia, type 2 diabetes, coronary heart disease, gallbladder disease, osteoarthritis, dental caries, gastrointestinal disorders, malignant neoplasms, and musculoskeletal disorders.

23. A prebiotic comprising the composition of claim 1.

24. A nutritional supplement comprising the composition of claim 1.

25. A comestible product comprising the composition of claim 1 wherein the composition is present in an amount equivalent to an amount of table sugar utilized in an otherwise similar composition.

26. The product of claim 25 wherein the composition of claim 1 comprises maltitol.

27. A formulation comprising the composition of claim 1 and an artificial sweetener.

28. The formulation of claim 27 wherein the artificial sweetener comprises acesulfame potassium, aspartame, neotaine, sucralose, advantame, or combinations thereof.
